(19) United States
(12) Patent Application Publication
(10) Pub. No.: US 2025/0135165 A1
(43) Pub. Date: May 1, 2025

(54) PLUG AND FLOOD DELIVERY SYSTEM FOR THERAPEUTIC AGENT, AND RELATED BALLOON CATHETER

(71) Applicant: BARD PERIPHERAL VASCULAR, INC., Franklin Lakes, NJ (US)

(72) Inventors: Casey Rockwood, Tulsa, OK (US); Melissa Boyle, Phoenix, AZ (US); Sam Shreve, Scottsdale, AZ (US); Edgar Sanchez Garcia, Tempe, AZ (US); David Tze, Phoenix, AZ (US); Clint Solomon, Gilbert, AZ (US)

(21) Appl. No.: 18/684,229

(22) PCT Filed: Aug. 17, 2021
(86) PCT No.: PCT/US2021/046291

§ 371 Date: Feb. 16, 2024

(51) Int. Cl. A61M 25/10; A61M 25/00

(52) U.S. Cl. CPC A61M 25/1011; A61M 25/0043; A61M 25/104; A61M 2025/1052; A61M 2025/1084; A61M 2205/0216

(57) ABSTRACT

An apparatus for performing angioplasty on a lesion and applying a therapeutic agent thereto. The apparatus includes a catheter having an inflatable balloon, such as a non-compliant balloon, adapted to compress the lesion when inflated. At least one port adapted to deliver the therapeutic agent to adjacent the lesion, such as once the balloon is deflated and advanced past a location where the inflatable balloon compressed the lesion. Alternatively, one or more additional balloons, which may be compliant balloons located distally or proximally of the balloon for compressing the lesion, may be inflated to form a sealed chamber including the compressed lesion and the therapeutic agent once delivered via the at least one port. Related methods are also disclosed.

### BACKGROUND OF THE INVENTION

Dilatation catheters with expandable elements or balloons are often used to treat lesions in vessels. In order to improve the treatment effect and, in particular, help to prevent restenosis, dilatation of a stenosed vessel is sometimes done in connection with the use of an applied therapeutic agent, or drug for short. However, in procedures where a drug-coated balloon is used and either there are multiple lesions, or a lack of correspondence between a balloon working surface and a treatment area, treatment of all would require multiple interventions each using a separate balloon. Furthermore, geographic misalignment, or “miss” is sometimes a concern, whereby a mismatch between the location of the drug on the balloon and the desired treatment area results, which can again lead to multiple interventions to achieve the desired treatment effect.

Others in the past have proposed arrangements by which a therapeutic agent may be applied independently of the balloon in order to overcome the above issues. A known arrangement uses three balloons arranged in series on a single catheter, whereby the first and third balloons are inflated on opposite sides of a desired location for treatment, such as a stenosis or lesion, in an effort to isolate the location. A second, intermediate balloon of the same type as the first and third balloons may be used for dilatation of the stenosis. The desired therapeutic agent is then introduced to the chamber at the location for treatment including the stenosis.

While such an arrangement avoids the multiple intervention situation potentially required as a consequence of using a drug-coated balloon, it suffers from several limitations. For one, the first and third balloons may not properly seal with the interior wall of the associated vessel. This can permit undesirable leakage of the therapeutic agent. This arrangement may in some cases also result in less effective dilation of the stenosis. Thus, in the end, little improvement over the use of a conventional drug-coated balloon may be realized. In some instances, the use of a simpler arrangement with only a single balloon may also be desirable to reduce complexity and cost.

Accordingly, a need is identified for an arrangement that overcomes some or all of the foregoing limitations, and perhaps others that have yet to be discovered.

### SUMMARY OF THE INVENTION

An object of the disclosure is to provide a balloon catheter that provide an improved treatment effect by both performing angioplasty and also delivering a therapeutic agent in a controlled manner, without relying on a coated balloon and, thereby, avoiding the potential need for repeated interventions.

According to a first aspect of the disclosure, an apparatus for performing angioplasty on a lesion and applying a therapeutic agent thereto is disclosed. The apparatus comprises a catheter including a single inflatable balloon adapted to compress the lesion when inflated, the catheter further including at least one port adapted to deliver the therapeutic agent to adjacent the lesion once the balloon is deflated and advanced past a location where the single inflatable balloon compressed the lesion.

In one embodiment, the single expandable balloon is non-compliant (i.e., a balloon that maintains its size and shape in one or more directions when the balloon is inflated, and in such case also has a pre-determined surface area that remains constant during and after inflation, also has a pre-determined length and pre-determined diameter that each, or together, remain constant during and after inflation). The at least one port may be proximal of the balloon, and may be in fluid communication with a lumen of the catheter, such as for delivering the therapeutic agent to the compacted lesion. A vacuum may also be connected to the lumen.

A further aspect of the disclosure pertains to an apparatus for performing angioplasty on a lesion and applying a therapeutic agent thereto. The apparatus includes a catheter including a non-compliant inflatable balloon adapted to compress the lesion when inflated, the catheter further including at least one port adapted to deliver the therapeutic agent to adjacent the lesion once the non-compliant inflatable balloon is deflated and advanced past a location where the non-compliant inflatable balloon compressed the lesion.

In one embodiment, the at least one port is a first port proximal of the non-compliant inflatable balloon, and further including a second port distal of the non-compliant inflatable balloon. The at least one port may be in fluid communication with a lumen of the catheter, and a vacuum may be connected to the lumen.

The non-compliant inflatable balloon may comprise inelastic fibers. In this or other embodiments, the catheter further includes at least one compliant balloon (e.g., formed of an stretchable or elastic material), meaning the addition of fluid pressure causes further expansion beyond a nominal diameter, and includes semi-compliant balloons, which have a more limited degree of elasticity but thus still allow for expansion beyond a nominal diameter) adapted for sealing the vessel to prevent flow. For example, the catheter may further include a first compliant balloon adapted for sealing the vessel to prevent flow proximal of the lesion and a second compliant balloon adapted for sealing the vessel to prevent flow distal of the lesion.

Yet another aspect of the disclosure includes an apparatus for performing angioplasty on a lesion and delivering a therapeutic agent thereto. The apparatus comprises a catheter including a first inflatable balloon having a first degree of compliance and a second inflatable balloon having a second degree of compliance less than the first degree of compliance.

In one embodiment, the first inflatable balloon comprises a non-compliant balloon including inelastic fibers, and/or the second inflatable balloon comprises a compliant or semi-compliant balloon. The catheter may include at least one port for delivering a therapeutic agent adjacent to the first inflatable balloon. The second inflatable balloon may be proximal of the first inflatable balloon, and further including a third inflatable balloon distal of the first inflatable balloon. In one particular embodiment, the catheter includes a first port between the first inflatable balloon and the second inflatable balloon for delivering a therapeutic agent, and a second port between the first inflatable balloon and the third inflatable balloon for delivering a therapeutic agent.

According to another aspect of the disclosure, an apparatus for performing angioplasty on a lesion in a vessel is provided. The apparatus includes a catheter having a proximal compliant balloon adapted for sealing the vessel to prevent flow proximal of the lesion, a distal compliant balloon adapted for sealing the vessel to prevent flow distal of the lesion, and an intermediate non-compliant balloon adapted to compress the lesion when expanded. The non-compliant balloon may comprise inelastic fibers.

In one embodiment, the catheter further includes at least one first port for delivering a therapeutic agent to adjacent the lesion. The at least one first port may be located on the catheter between the distal compliant balloon and the intermediate non-compliant balloon, and may be in fluid communication with a lumen of the catheter. The catheter may further include at least one second port for delivering a therapeutic agent to adjacent the lesion, which may be connected to the same or a different lumen. The at least one second port may be located on the catheter between the proximal compliant balloon and the intermediate non-compliant balloon. A vacuum may be connected to the lumen.

Still a further aspect of the disclosure pertains to a method of performing angioplasty, comprising, in the following order: (1) inflating a balloon on a catheter to compress a lesion; (2) deflating the balloon; (2) moving the catheter such that a port therein is adjacent to the lesion; (3) inflating the balloon with the catheter moved; and (4) delivering a therapeutic agent to the lesion via the port in the catheter. The method may further include the step of evacuating any residual therapeutic agent via the port, which may be proximal of the balloon.

Yet another aspect of the disclosure pertains to a method of performing angioplasty. The method comprises inflating a proximal compliant balloon adapted for sealing the vessel to prevent flow proximal of the lesion, inflating a distal compliant balloon adapted for sealing the vessel to prevent flow distal of the lesion, and inflating an intermediate non-compliant balloon adapted to compress the lesion when expanded. The method may further include the steps of deflating the intermediate non-compliant balloon while the proximal compliant balloon and distal compliant balloon remain inflated for sealing the vessel, and delivering a therapeutic agent to adjacent the lesion. The method may further include the step of evacuating any residual therapeutic agent.

### BRIEF DESCRIPTION OF THE DRAWINGS

The above and further advantages of the present disclosure may be better understood by referring to the following description in conjunction with the accompanying drawings in which:

FIG. 1 is side view of a balloon catheter according to one aspect of the disclosure;

FIG. 2 is a cutaway view of the balloon, and FIG. 2A is a cross-sectional view along line 2A-2A of FIG. 2;

FIGS. 3, 4, and 5 are progressive views illustrating a use of the balloon catheter of FIG. 1;

FIG. 6 is side view of a balloon catheter according to one aspect of the disclosure, and FIG. 6A is a cross-sectional view along line 6A-6A of FIG. 6; and

FIGS. 7, 8, and 9 are progressive views illustrating use of the balloon catheter of FIG. 6.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the present inventive concepts.

The invention is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

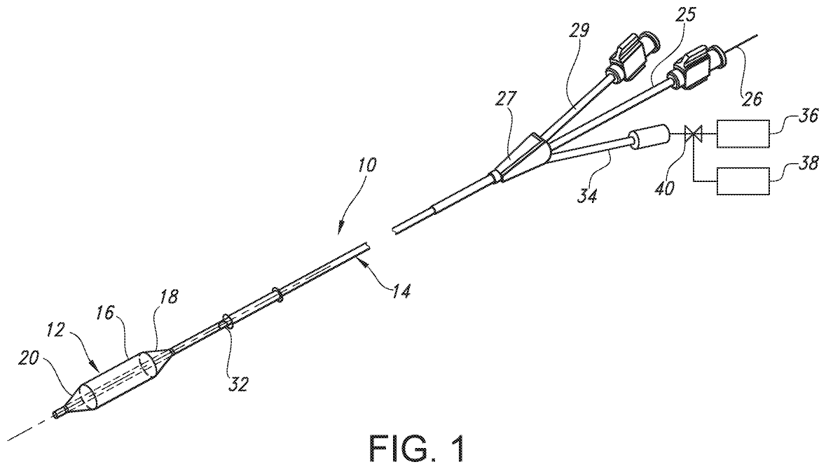

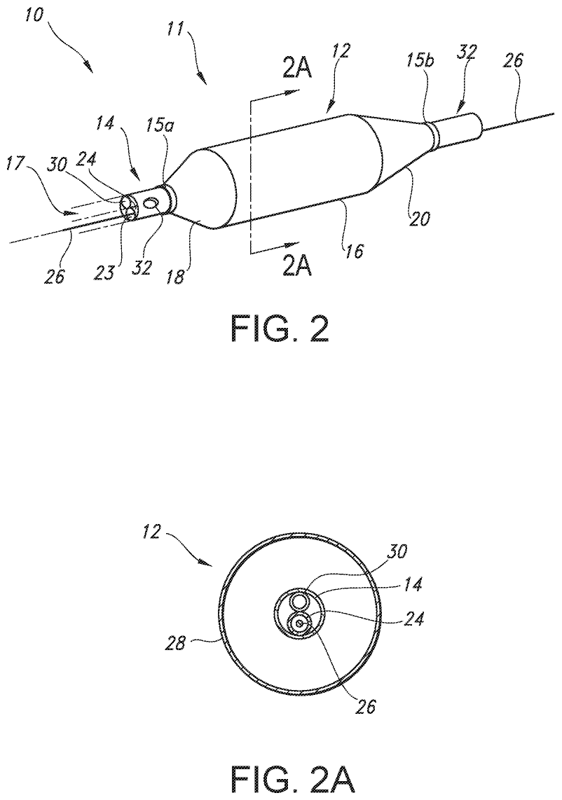

With reference to FIGS. 1 and 2, provided is a catheter 10 having a distal end portion 11 (isolated in FIG. 2) including an expandable element in the form of a single balloon 12 mounted on a catheter shaft 14. The balloon 12 includes an intermediate portion 16, or “barrel,” and end portions 18, 20. In one embodiment, the end portions 18, 20 reduce in diameter or taper to join the intermediate portion 16 to the catheter shaft 14 (and thus portions 18, 20 are generally termed cones or cone portions). The balloon 12 is sealed at balloon ends (proximal end 15a and distal end 15b) on the cone portions 18, 20 to allow the inflation of the balloon 12 via one or more inflation lumens 17 extending within catheter shaft 14 and communicating with the interior of the balloon 12.

The catheter shaft 14 further includes an elongated, tubular shaft 24 at least partially nested therein. This internal shaft 24 includes a guidewire lumen that directs a guidewire 26 through the catheter 10, including along the distal end portion 11 on which the single balloon 12 is located, and out through a tip of the catheter. As illustrated in FIG. 2, this guidewire 26 may extend through the proximal end portion of the catheter 10 and through a first port 25 of a connector or hub 27 into the guidewire lumen to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown). A second port 29 may also be associated with catheter 10, such as by way of connector or hub 27, for introducing a fluid (e.g., saline solution, a contrast agent, or both) into the interior compartment of the balloon 12 via the inflation lumen 17.

Inflatable balloon 12 may include a single or multi-layered balloon wall 28 forming the interior for receiving the inflation fluid. The balloon 12 may be a non-compliant balloon having a balloon wall 28 that maintains its size and shape in one or more directions when the balloon is inflated, and may comprise a plurality of inelastic fibers arranged in one or more layers. The balloon 12 in such case also has a pre-determined surface area that remains constant during and after inflation, also has a pre-determined length and pre-determined diameter that each, or together, remain constant during and after inflation. The balloon 12 (as well as any balloon described herein) can have any of a variety of diameters, ranging from 1.25-40 mm or 2.0-8.0 mm, and may have any of a variety of lengths such as 10-300 mm or 20-300 mm. As outlined further in the discussion that follows, more than one inflatable balloon may be associated with the catheter.

According to a first aspect of the disclosure, the catheter 10 may be adapted to deliver a therapeutic agent or drug to a treatment location, independent of the balloon (that is, not as a coating on the balloon). This delivery may be achieved by providing the catheter 10 with a lumen 30 for delivering the therapeutic agent to one or more ports 32 formed in the shaft 14. The one or more ports 32 may be located proximal of the balloon 12, or instead may be distal of the balloon.

A distal end of the lumen 30 may communicate with a third port 34 associated with the connector or hub 27, and may be attached to a source 36 of a therapeutic agent (such as, for example, a syringe including the selected agent, such as Paclitaxel, as one example). The lumen 30 may also be in communication with a source of vacuum 38 (which may be the same syringe delivering the therapeutic agent). If a distinct device, vacuum source 38 may be connected via a common delivery line with the therapeutic agent source 36 using a valve 40.

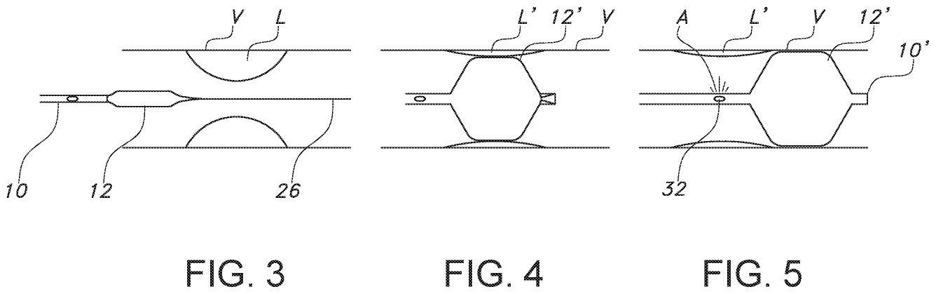

Turning now to FIGS. 3, 4, and 5, an exemplary use of the catheter 10 is shown and described. As indicated in FIG. 3, using the guidance of a guidewire 26, the catheter 10 may be advanced to a treatment location, such as a stenosis or lesion L in a vessel V. The balloon 12 may then be inflated to dilate the lesion L, as shown in FIG. 4 (note inflated balloon 12′). After the lesion L has been dilated, the balloon 12 is at least partially deflated, the catheter 10 tracked to a location spaced to the site of the lesion L, such as by advancing, and reinflated (note balloon 12′) to block blood flow downstream temporarily, and thus form a plug, as shown in FIG. 5.

While fluid (e.g., blood) flow is halted temporarily, the therapeutic agent A is then delivered to the site via lumen 30 through port(s) 32 proximal of the balloon 12, but now generally aligned with the compacted lesion L′ in view of the advance of the catheter 10. The agent A thus floods in the plugged area adjacent to the lesion L for a set amount of time or set volume, which may be selected by the clinician depending on the desired treatment regimen. Thereafter, a vacuum may be applied to the port 32 (such as via source 38) to withdraw any remaining therapeutic agent through the same dispersal lumen 30. The inflated balloon 12′ may then be deflated and catheter 10 withdrawn, or else redeployed or repositioned for a further treatment by repeating the above-mentioned steps.

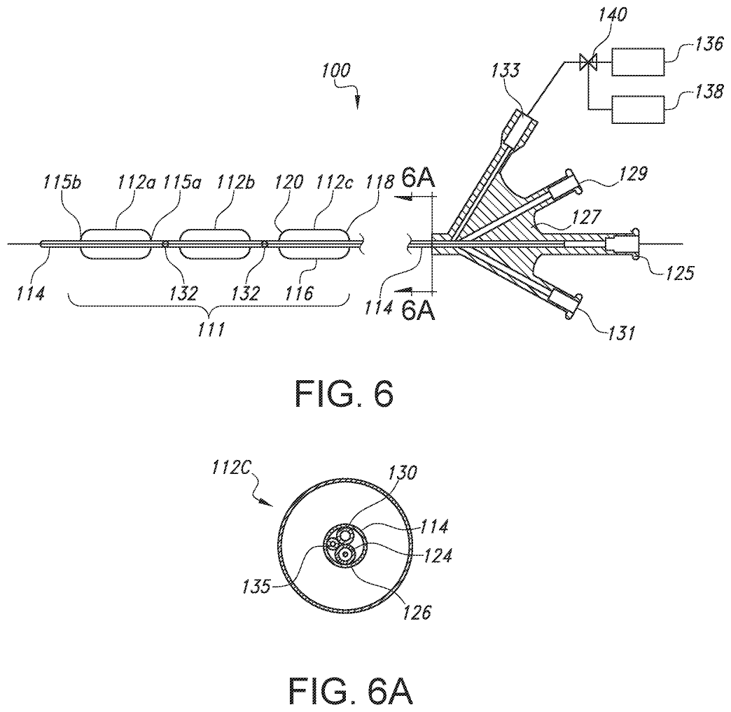

With reference to FIG. 6, an alternative embodiment of a catheter 100 is shown having a distal end portion 111 with a plurality of balloons 112a, 112b, 112c mounted in tandem on a catheter shaft 114. Each balloon 112a, 112b, 112c may be of similar shape. Thus, with reference only to balloon 112c, each balloon may have an intermediate portion 116, or “barrel,” and end portions 118, 120. In one embodiment, the end portions 118, 120 reduce in diameter to join the intermediate portion 116 to the catheter shaft 114 (and thus portions 118, 120 are generally termed cones or cone portions). Each balloon 112a, 112b, 112c is sealed at balloon ends (proximal end 115a and distal end 115b of balloon 112a) on the cone portions 118, 120.

The shaft 114 of catheter 100 also includes an elongated, tubular internal shaft 124. This internal shaft 114 includes a guidewire lumen that directs a guidewire 126 through the catheter 100, and along the distal end portion of which the balloons 112a, 112b, 112c may be located. This guidewire 126 may extend through the proximal end portion of the catheter 100 and through a connector or hub 127 via port 125 into a lumen to achieve an “over the wire” (OTW) arrangement. However, as with catheter 10, catheter 100 could also be provided in a “rapid exchange” (RX) configuration (in which the guidewire 126 exits a lateral opening closer to the distal end, not shown) or else is fed through the tip at a passage distally of the balloons (“short” RX; not shown).

Additional ports 129, 131 may also be associated with catheter 100, such as by way of the connector or hub 127, for introducing a fluid (e.g., saline solution, a contrast agent, or both) into the interior compartment of the balloons 112a, 112b, 112c. Specifically, the first port 129 may be associated with a lumen 133 for delivering fluid concurrently to the first and third balloons 112a, 112c via associated outlets in shaft 114 communicating with the interior compartments, while the second port 131 delivers fluid to the second or intermediate balloon 112b via a lumen 135, which again may communicate with the interior compartment thereof.

According to this second aspect of the disclosure, the catheter 100 may also optionally be adapted to deliver a therapeutic agent or drug to a treatment location, such as a stenosis, independent of the balloon (that is, not as a coating on the balloon). This delivery may be achieved by providing the catheter 100 with a lumen 130 for delivering the therapeutic agent to one or more ports 132 in the shaft 114, which may be located between the first (distal) and third (proximal) balloons 112a, 112c, such as on either side of the second (intermediate) balloon 112b. A proximal end of the lumen 130 may communicate with a port 133 associated with the connector or hub 127, and may be attached to a source 136 of a therapeutic agent (such as, for example, a syringe including a pharmaceutical, such as Paclitaxel as one example). The lumen 130 may also be in communication with a source of vacuum 138 (which could be the same syringe serving to deliver the therapeutic agent). If a distinct device, it may be connected via a common delivery line with the therapeutic agent source 136 using a valve 140.

In this embodiment, the balloons 112a, 112b, 112c may also be provided with different levels of compliance. For example, the first and third balloons 112a, 112c may be compliant (e.g., formed of an stretchable or elastic material), meaning the addition of fluid pressure causes further expansion beyond a nominal diameter (and includes semi-compliant balloons, which have a more limited degree of elasticity but thus still allow for expansion beyond a nominal diameter). The second balloon 112b may be non-compliant (e.g., formed of inelastic materials, such as fibers), which means that the additional fluid pressure does not cause any appreciable expansion beyond a nominal diameter. More specifically, once the non-compliant balloon 112b is fully inflated, it has a length and diameter that do not change as the pressure on the interior of the balloon 112b increases.

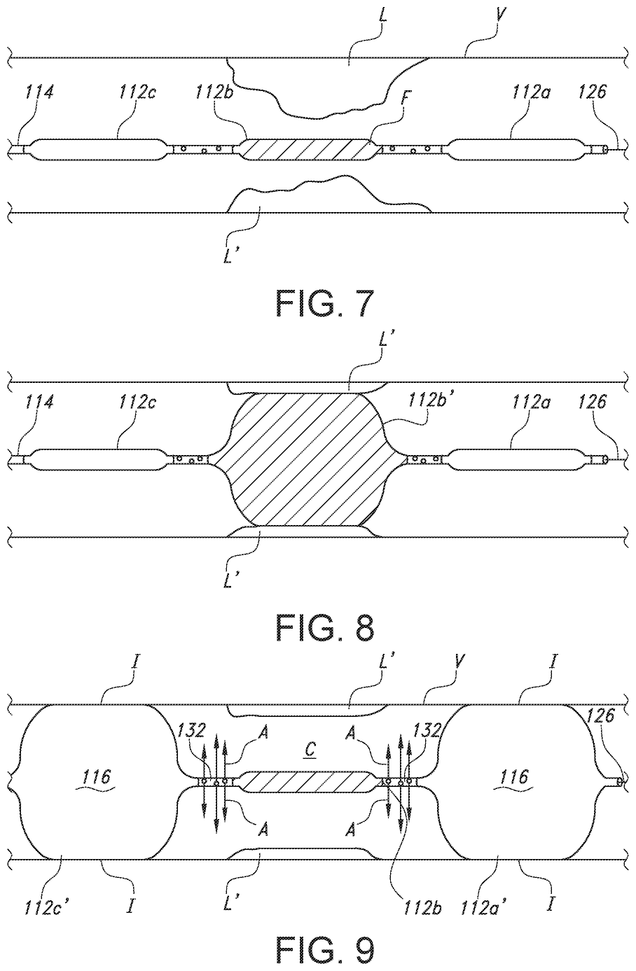

Thus, in one possible use, the catheter 100 according to the second embodiment of FIGS. 7, 8, and 9, may be advanced along a guidewire 126 within a vessel V such that the second balloon 112b in a deflated condition is adjacent to a treatment area, such as a lesion L. The second balloon 112b may then be expanded (note inflated balloon 112b′) and, in view of the non-compliant characteristics (note fibers F), used to dilate or compact the lesion (note compacted lesion L′ in FIG. 8).

The second balloon 112b may then be deflated, as shown in FIG. 9, and the first and third balloons 112a, 112c expanded (note inflated balloons 112a′, 112c′). In view of the compliant characteristics of these balloons 112a, 112c, inflation thus allows the outer surface of the balloons and, particularly, the barrel section 116, to establish close contact with the interior wall I of the vessel V, thereby forming a seal. The therapeutic agent A may then be delivered to the sealed chamber C thus formed between the inflated balloons 112a′, 112c′, which includes the compacted lesion L′, and provide the intended pharmaceutical effect. The agent A may be provided for a set amount of time or a set volume, which may be selected by the clinician depending on the desired treatment regimen. Thereafter, a vacuum may be applied to the dispersal lumen 130 associated with ports 132 (such as via source 138) to withdraw any remaining therapeutic agent. The inflated balloon 112b may then be deflated, and the catheter 100 withdrawn from the vessel, or repositioned for a further treatment, if desired.

In terms of materials, noncompliant (high-pressure) balloons are typically made of polyester or nylon. Semi compliant (midpressure) balloons are commonly made of Pebax or higher-durometer polyurethanes. Compliant (elastomeric) balloons are typically made of polyurethane or silicone. However, the foregoing materials are simply examples, and the disclosed concepts are not considered to be limited to any particular materials or manner of fabrication.

Summarizing, this disclosure may be considered to relate to the following items:
1. An apparatus for performing angioplasty on a lesion and applying a therapeutic agent thereto, comprising:
   a catheter including a single inflatable balloon adapted to compress the lesion when inflated, the catheter further including a port adapted to deliver the therapeutic agent to adjacent the lesion once the balloon is deflated and moved away from a location where the single inflatable balloon compressed the lesion.
2. The apparatus of item 1, wherein the single inflatable balloon is non-compliant.
3. The apparatus of item 1 or item 2, wherein the port is proximal of the single inflatable balloon.
4. The apparatus of any of items 1-3, wherein the port is in fluid communication with a lumen of the catheter.
5. The apparatus of claim 4, further including a vacuum connected to the lumen.
6. An apparatus for performing angioplasty on a lesion and applying a therapeutic agent thereto, comprising:
   a catheter including a non-compliant inflatable balloon adapted to compress the lesion when inflated, the catheter further including at least one port adapted to deliver the therapeutic agent to adjacent the lesion once the non-compliant inflatable balloon is deflated and moved from a location where the non-compliant inflatable balloon compressed the lesion.
7. The apparatus of item 6, wherein the at least one port is a first port proximal of the non-compliant inflatable balloon, and further including a second port distal of the non-compliant inflatable balloon.
8. The apparatus of item 6, wherein the at least one port is in fluid communication with a lumen of the catheter.
9. The apparatus of item 6, further including a vacuum connected to the lumen.
10. The apparatus of any of items 6-9, wherein the non-compliant inflatable balloon comprises inelastic fibers.
11. The apparatus of any of items 6-10, wherein the catheter further includes at least one compliant balloon adapted for sealing the vessel to prevent flow.
12. The apparatus of item 6, wherein the catheter further includes a first compliant balloon adapted for sealing the vessel to prevent flow proximal of the lesion and a second compliant balloon adapted for sealing the vessel to prevent flow distal of the lesion.
13. An apparatus for performing angioplasty on a lesion and delivering a therapeutic agent thereto, comprising:
   a catheter including a first inflatable balloon having a first degree of compliance and a second inflatable balloon having a second degree of compliance less than the first degree of compliance.
14. The apparatus of item 13, wherein the first inflatable balloon comprises a non-compliant balloon including inelastic fibers.
15. The apparatus of item 14, wherein the second inflatable balloon comprises a compliant or semi-compliant balloon.
16. The apparatus of any of items 13-15, wherein the catheter includes at least one port for delivering a therapeutic agent adjacent to the first inflatable balloon.
17. The apparatus of any of items 13-16, wherein the second inflatable balloon is proximal of the first inflatable balloon, and further including a third inflatable balloon distal of the first inflatable balloon.
18. The apparatus of any of items 13-17, wherein the catheter includes a first port between the first inflatable balloon and the second inflatable balloon for delivering a therapeutic agent, and a second port between the first inflatable balloon and the third inflatable balloon for delivering a therapeutic agent.
19. An apparatus for performing angioplasty on a lesion in a vessel, comprising:
   a catheter including a proximal compliant balloon adapted for sealing the vessel to prevent flow proximal of the lesion, a distal compliant balloon adapted for sealing the vessel to prevent flow distal of the lesion, and an intermediate non-compliant balloon adapted to compress the lesion when expanded.
20. The apparatus of item 19, the catheter further including at least one first port for delivering a therapeutic agent to adjacent the lesion.
21. The apparatus of item 19, wherein the at least one first port is located on the catheter between the distal compliant balloon and the intermediate non-compliant balloon.
22. The apparatus of item 20 or item 21, the catheter further including at least one second port for delivering a therapeutic agent to adjacent the lesion.
23. The apparatus of item 22, wherein the at least one second port is located on the catheter between the proximal compliant balloon and the intermediate non-compliant balloon.
24. The apparatus of item 22 or item 23, wherein the at least one first port and the at least one second port are in fluid communication with a lumen of the catheter.
25. The apparatus of item 24, further including a vacuum connected to the lumen.
26. The apparatus of any of items 19-25, wherein the non-compliant balloon comprises inelastic fibers.
27. A method of performing angioplasty on a lesion in a vessel and delivering a therapeutic agent thereto, comprising, in order:
   inflating a balloon on a catheter to compress the lesion;
   deflating the balloon;
   moving the catheter to a location such that a port in the catheter is adjacent to the lesion;
   inflating the balloon; and
   delivering the therapeutic agent to the lesion via the port.
28. The method of item 27, further including the step of evacuating any residual therapeutic agent via the port.
29. The method of item 27, wherein the moving step comprises advancing the catheter.
30. A method of performing angioplasty on a lesion in a vessel, comprising:
   inflating a proximal compliant balloon adapted for sealing the vessel to prevent flow proximal of the lesion;
   inflating a distal compliant balloon adapted for sealing the vessel to prevent flow distal of the lesion; and
   inflating an intermediate non-compliant balloon adapted to compress the lesion when expanded.
31. The method of item 30, further including the steps of:
   deflating the intermediate non-compliant balloon while the proximal compliant balloon and distal compliant balloon remain inflated for sealing the vessel; and
   delivering a therapeutic agent to adjacent the lesion.
32. The method of item 30, further including the step of evacuating any residual therapeutic agent.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. For example, for the three balloon embodiment, one of the first or third balloons may be omitted, and this or both of the balloons may be made compliant, semi-compliant, or non-compliant. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1-11. (canceled)

12. The apparatus of claim 6, wherein the catheter further includes a first compliant balloon adapted for sealing the vessel to prevent flow proximal of the lesion and a second compliant balloon adapted for sealing the vessel to prevent flow distal of the lesion.

13. An apparatus for performing angioplasty on a lesion and delivering a therapeutic agent thereto, comprising:
a catheter including a first inflatable balloon having a first degree of compliance and a second inflatable balloon having a second degree of compliance less than the first degree of compliance.

14. The apparatus of claim 13, wherein the first inflatable balloon comprises a non-compliant balloon including inelastic fibers.

15. The apparatus of claim 14, wherein the second inflatable balloon comprises a compliant or semi-compliant balloon.

16. The apparatus of claim 13, wherein the catheter includes at least one port for delivering a therapeutic agent adjacent to the first inflatable balloon.

17. The apparatus of claim 13, wherein the second inflatable balloon is proximal of the first inflatable balloon, and further including a third inflatable balloon distal of the first inflatable balloon.

18. The apparatus of claim 13, wherein the catheter includes a first port between the first inflatable balloon and the second inflatable balloon for delivering a therapeutic agent, and a second port between the first inflatable balloon and the third inflatable balloon for delivering the therapeutic agent.

19. An apparatus for performing angioplasty on a lesion in a vessel, comprising:
a catheter including a proximal compliant balloon adapted for sealing the vessel to prevent flow proximal of the lesion, a distal compliant balloon adapted for sealing the vessel to prevent flow distal of the lesion, and an intermediate non-compliant balloon adapted to compress the lesion when expanded.

20. The apparatus of claim 19, the catheter further including at least one first port for delivering a therapeutic agent to adjacent the lesion.

21. The apparatus of claim 19, wherein the at least one first port is located on the catheter between the distal compliant balloon and the intermediate non-compliant balloon.

22. The apparatus of claim 21, the catheter further including at least one second port for delivering a therapeutic agent to adjacent the lesion.

23. The apparatus of claim 22, wherein the at least one second port is located on the catheter between the proximal compliant balloon and the intermediate non-compliant balloon.

24. The apparatus of claim 22, wherein the at least one first port and the at least one second port are in fluid communication with a lumen of the catheter.

25. The apparatus of claim 24, further including a vacuum source connected to the lumen.

26. The apparatus of claim 21, wherein the non-compliant balloon comprises inelastic fibers.

27. A method of performing angioplasty on a lesion in a vessel and delivering a therapeutic agent thereto, comprising, in order:
inflating a balloon on a catheter to compress the lesion;
deflating the balloon;
moving the catheter to a location such that a port in the catheter is adjacent to the lesion;
inflating the balloon; and
delivering the therapeutic agent to the lesion via the port.

28. The method of claim 27, further including the step of evacuating any residual therapeutic agent via the port.

29. The method of claim 27, wherein the moving step comprises advancing the catheter.

30. A method of performing angioplasty on a lesion in a vessel, comprising:
inflating a proximal compliant balloon adapted for sealing the vessel to prevent flow proximal of the lesion;
inflating a distal compliant balloon adapted for sealing the vessel to prevent flow distal of the lesion; and
inflating an intermediate non-compliant balloon adapted to compress the lesion when expanded.

31. The method of claim 30, further including the steps of:
deflating the intermediate non-compliant balloon while the proximal compliant balloon and distal compliant balloon remain inflated for sealing the vessel; and
delivering a therapeutic agent to adjacent the lesion.

32. The method of claim 30, further including the step of evacuating any residual therapeutic agent.
